(19) United States
(12) Patent Application Publication
(10) Pub. No.: US 2006/0002878 A1
(43) Pub. Date: Jan. 5, 2006

(54) Cosmetic article and related methods

(75) Inventors: David Acher, Hull (GB); Frederic De La Torre, Hull (GB)

Correspondence Address: NORRIS, MCLAUGHLIN & MARCUS, 875 THIRD AVE, 18TH FLOOR, NEW YORK, NY, 10022, US

(73) Assignee: Reckitt Benckiser (UK) Limited, Slough, Berkshire (GB)

(21) Appl. No.: 10/523,945

(22) PCT Filed: Aug. 1, 2003
(86) PCT No.: PCT/GB03/03346

§ 371 Date: Feb. 8, 2005

(30) Foreign Application Priority Data
Aug. 10, 2002 (GB) 0218605.4

(51) Int. Cl. A61K 7/06; A61K 7/11; A61K 7/15

(52) U.S. Cl. 424/70.11; 424/73

(57) ABSTRACT

The present invention provides a depilatory patch comprising a layer of depilatory composition carried by a substrate. The depilatory patch is arranged, in use, to be applied to the skin of a user and left for a prescribed time period before being removed. The depilatory composition acts to degrade or destroy hair on the skin where the patch has been applied. The invention also extends to methods of manufacturing depilatory patches of the invention, and to depilatory methods using such patches.

This invention relates to hair removing products, to their manufacture and to methods of hair removal.

It is known to provide depilatory compositions in the form of lotions, creams or the like, which are applied to the skin with a spatula or other implement. After being left on the skin for a desired period of time the composition is removed, either by scraping or peeling the composition from the skin. The removed composition includes embedded hair debris where the hair has been chemically broken down, leaving a hairless region on the skin. In use, the composition is generally applied as a relatively thick coating, to the area where it is wished to remove hair, such as the legs or the axilla, and left in situ for around 5 to 10 minutes, for the hair to be chemically broken down. The composition is formulated so as to have a sufficiently high viscosity to prevent its flow away from the area to which it has been applied.

The operation of applying the depilatory composition, especially when in a paste or cream formulation, and removing it can be time consuming and awkward. Frequently, the composition has to be scraped carefully from the skin in order to remove all of the composition. This operation can irritate the skin and take a considerable length of time. Prolonged contact of the composition with the skin can itself irritate the skin.

Furthermore when a lotion, cream or the like is applied to certain areas of the body, such as the axilla, the user must be very careful to prevent the lotion or cream from being transferred to other parts of the body, or to clothing.

It would be preferable to provide a depilatory composition which needs minimal manipulation by a user, which is easy to apply accurately to the skin and which may be removed efficiently and with minimal effort by the user after the hair has been degraded.

It is known to provide epilatory compositions formed of viscoelastic materials. The viscoelastic materials may in certain embodiments be rosin-based. In other embodiments they may be sugar-based.

In some products the epilatory compositions may be supplied in the form of strips, retained between cellophane sheets. A box of several sheets is typically purchased. The cellophane sheets may have coatings of polyvinyl chloride, which acts as a barrier preventing the composition, or components of it, from migrating through the sheets; and also having the correct release properties for use. In use, the user peels away one of the cellophane sheets, presses the epilatory strip firmly onto the area to be plucked using the other cellophane sheet, then removes that sheet. The user then presses a strip, of fabric onto the epilatory strip, then pulls one end of the fabric sharply away from the skin. The hairs trapped in the composition are removed from the treated area along with, optimally, all of the composition, still attached to the fabric.

The use of epilatory strips has been successful but is not free of problems. One problem that has been reported is that of the epilatory composition flowing from between the sheets in very warm weather, so that a customer purchasing a box of strips may encounter a messy, unusable mass of the hair removing composition, still fluid or re-set, in the box.

By “epilatory” herein we mean able mechanically to remove hairs which are still intact, whether by breaking them where they are thinned or by pulling them out by their roots.

By “depilatory” we mean able to degrade or dissolve hair, by chemical action.

Preferred embodiments of the present invention seek primarily to solve the aforementioned problems with existing products, in particular to provide easier application and removal of the compositions from the skin than existing depilatory compositions, yet without the leakage problems associated with prior epilatory compositions in the form of strips.

According to a first aspect of the present invention there is provided a packaged hair removing product comprising a container and a hair-removing layer therein, wherein the container comprises a sheet formed with a shallow well, the hair-removing layer being retained in the shallow well, the container further comprising a closure member which retains the hair-removing layer in the container, the closure member being removable or displaceable from the container to permit removal of the hair-removing layer.

The composition may be in the form of a strip. The container may be sufficiently flexible for the strip to be applied to the skin, after removal or displacement of the closure member, whilst still in its shallow well. Alternatively it may, for example, be in contact with a release sheet of the form used in the prior art, thereby to facilitate application.

Preferably, however, the layer is carried by a substrate, the layer and the substrate being substantially inseparable, so as to form a “patch”. Preferably the substrate protrudes beyond the layer. Suitably the substrate is dimensioned to protrude laterally from substantially the entire periphery of the hair-removing layer.

By “patch” we mean an article comprising a substrate on which is carried a layer of hair-removing composition, which layer is designed to remain in contact with the substrate during application, use and removal of the article.

The use of a packaged product to supply a hair-removing composition as a layer in a well, mitigates or eliminates the prior problems described above. A layer of hair-removing composition is supplied, preferably as a self-supporting body, not as a lotion or cream, which may be messy to apply and may be spread to parts of body other than that from which it is desired to remove hair. It is packaged within a well and retained between the sheet in which the well is formed, and the closure member. The risk of migration of the composition is thereby at the least substantially reduced, and preferably eliminated.

Preferably the recess is stepped, and has an inner, deeper region in which the layer is located and an adjoining, shallower region, in which projecting portion(s) of the substrate is/are located.

Preferably, therefore, the packaged hair-removing product comprises a container and a hair-removing patch therein, the hair-removing patch having a layer of hair-removing composition, carried on a substrate which projects laterally from at least a portion of the layer of hair-removing composition, wherein the container comprises a sheet formed with a recess, the recess having an inner region in which the layer of hair-removing composition is located and an outer region which abuts but is shallower than the inner region, and in which the lateral portion of the substrate is located, the package further comprising said closure member.

Suitably the substrate is a flexible sheet material capable of carrying the layer of depilatory composition. The sheet material may be porous, absorbent and/or fibrous such that the depilatory composition is partially absorbed into the carrier to provide anchorage of the layer on the substrate. Suitably layer and substrate are bonded such that one cannot be peeled from the other.

The substrate may be woven or non-woven. The substrate may comprise natural or synthetic material or mixtures thereof.

Suitable natural substrate materials include cellulosic material, such as cellulose per se or derived from wood pulp, cotton, hemp, jute, flax and fibre mixtures thereof, for example.

Suitable synthetic substrates include rayon, polyesters, polyurethanes, polyvinyl acetate, polyacrylates, polymethacrylates, polyamides, styrenes, styrene co-polymers, polyolefins, polyvinylchlorides, inorganic fibres, polyethylene, polypropylene, polyethylene terephthalate, nylons, and mixtures and co-polymers thereof, for example.

The substrate may comprise more than one layer of sheet material. Each layer of sheet material may be constructed from the same or different materials. Suitably each layer is laminated to an adjacent layer.

Suitably the substrate on which the layer may suitably be carried comprises a non-woven sheet material and is preferably a non-woven polyethylene layer.

Suitably the shape of a substrate employed corresponds to the shape of the hair-removing layer, although preferably not to size. Alternatively the shape of the substrate may be different to the shape of the layer.

Suitably, the layer of hair-removing composition is a layer of generally even thickness, preferably always within ±10% of the mean thickness.

Suitably the ratio of the smallest diameter of the layer to its mean thickness is at least 20, preferably at least 40.

Suitably the layer of hair-removing composition has a thickness of between 0.5 mm and 2.0 mm, preferably between 1.0 mm and 1.5 mm and especially substantially 1.2 mm.

Suitably the layer is of a self-supporting body substantially without propensity to migrate from the locus to which it is applied, even when vertical, and removed from the package and carried by the skin, and thus somewhat warmed.

The layer of hair-removing composition may be of any size and geometry, and may particularly be round, oval, rectangular, square, L-shaped, T-shaped, semi-circular, crescent-shaped, U-shaped or V-shaped. The shape of the layer may be determined by the body area to which it is intended to be connected to in use. Thus, for example, for patches intended for hair removal from the axilla, an oval (including elliptical) shaped layer is preferable. For patches intended for hair removal from the upper lip a crescent shaped layer is preferred.

Preferably the minimum diameter of an oval axilla patch is at least 40 mm, more preferably at least 50 mm, most preferably at least 60 mm.

Preferably the minimum diameter of an oval axilla patch is up to 90 mm, more preferably up to 80 mm, most preferably up to 70 mm.

Preferably the maximum diameter of an oval axilla patch is up to 170 mm, more preferably up to 160 mm, most preferably up to 150 mm.

Preferably the maximum diameter of an oval axilla patch is at least 120 mm, more preferably at least 130 mm, most preferably at least 140 mm.

The layer may comprise a peelable release layer connected to one side of the hair-removing layer. The other side may have a further peelable release layer or a said substrate, when the product is a patch. The release layer may comprise a peelable silicone sheet, or a peelable plastics sheet.

Suitably the hair-removing composition comprises one or more depilatory agents which degrade and/or destroy hair. Suitable depilatory agents include sulphur compounds such as potassium thioglycolate, dithioerythritol, thioglycerol, thioglycol, thioxanthine, thiosalicylic acid, N-acetyl-L-cysteine, lipoic acid, NaHSO₃, Li₂S, Na₂S, K₂S, MgS, CaS, SrS, BaS, (NH₄)₂S, sodium dihydrolipoate 6,8-dithiooctanoate, sodium 6,8-dithiooctanoate, salts of hydrogen sulphide for example NaSH or KSH, thioglycolic acid, thioglycerol, 2-mercaptopropionic acid, 3-mercaptopropionic acid, thiomalic acid, ammonium thioglycolate, glyceryl monothioglycolate, monoethanolamine thioglycolate, monoethanolamine thioglycolic acid, diammonium dithiodiglycolate, ammonium thiolactate, monoethanolamine thiolactate, thioglycolamide, homocysteine, cysteine, glutathione, dithiothreitol, dihydrolipoic acid, 1,3-dithiopropanol, thioglycolamide, glyceryl monothioglycolate, thioglycolhydrazide, keratinase, hydrazine sulphate, hydrazine disulphate, triisocyanate, guanidine thioglycolate, calcium thioglycolate and/or cysteamine. Preferably a depilatory composition comprises a thioglycolate, more preferably potassium thioglycolate, as a depilatory agent.

Suitably a depilatory agent is provided in the depilatory composition in an amount of between 1 wt % and 10 wt %, preferably between 1.5 wt % and 8 wt % and more preferably between 2 wt % and 6 wt % of the total weight of the composition.

Optionally a depilatory composition includes an accelerator that accelerates the rate of depilatory action of the depilatory agent, such as urea, thiourea, dimethyl isosorbide (DMI), ethoxydiglycol (Transcutol) or methylpropyldiol (Mp diol). The depilatory composition preferably comprises from 5 wt % to 15 wt %, more preferably from 6 wt % to 10 wt % of an accelerator, when present.

Alternatively or additionally the hair-removing composition is epilatory. Thus, the hair-removing composition may comprise a matrix or polymeric material, which suitably has properties which may be described as gel-like, waxy or viscous.

Preferably, the viscoelastic properties of the composition do not change substantially as a function of time, or by addition of a chemical compound. Preferably they do change, from viscous-dominated to elastic-dominated, as a function of frequency of applied stress.

Thus, the composition may comprise a polymeric material comprising a tackifying epilatory material, for example a tackifying aliphatic or aromatic resin, ethylene vinyl acetate, styrene-butene-styrene, a tackifying block copolymer, or a mixture of polyvinyl alcohol and a gel-promoting agent, preferably boric acid, for example. Suitable tackifying resins include rosinaceous materials, for example a rosin ester and/or colophony.

Preferably the composition comprising polyvinyl alcohol is formulated to be a tacky viscous or gel-like material, at ambient temperature, not a rubber-like material or a material which sets in use to form a rubber-like material, in an unstressed condition, at ambient temperature. Preferably the composition is formulated to become elastic on being abruptly stressed, at body temperature, so that it may be peeled from the skin as a sheet. Preferably the composition is formulated to be tacky, at ambient temperature.

Preferably, the polyvinyl alcohol is of molecular weight Mw at least at least 1,000 g/mol, more preferably at least 10,000 g/mol, and most preferably at least 20,000 g/mol (mean weights of the molar masses determined by Gel Permeation Chromatography).

Preferably the polyvinyl alcohol is of molecular weight Mw up to 500,000 g/mol, more preferably up to 200,000 g/mol, and most preferably up to 40,000 g/mol (determined as above).

Preferably the polyvinyl alcohol is hydrolysed to a degree of hydrolysis of at least 70%, more preferably at least 87%, and most preferably at least 97%.

Suitably the polyvinyl alcohol is present in an amount of between 0.1 wt % and 25 wt %, preferably between 1 wt % and 15 wt %, more preferably between 2 wt % and 10 wt % and most preferably between 4 wt % and 8 wt % of the total weight of the composition.

Preferably a cosmetically-acceptable gel-promoting agent is employed with the polyvinyl alcohol.

A preferred class of gel-promoting agent is boron-containing compounds, especially acids and salts, for examples borates and boric acid. Favoured borates include perborates, metaborates and tetraborates (e.g. borax). Most preferred as a gel-promoting agent is boric acid.

Another suitable class of gel-promoting agent is organic azo dyes, for example Congo Red (sodium diphenyldiazo-bis-naphthylamine-sulphonate), benzopurpurine 4B, Congo Corinth G and benzoazurine G.

Other suitable gel-promoting agent may include: germanic acid and germanates; titanium salts and esters, particularly titanium IV-triethanolamine; chromates; vanadates; Group IB metal salts, particularly cupric salts; mono- and di-aldehydes (e.g. glutaraldehyde); dicarboxylic acids (e.g. maleic acid, oxalic acid, malonic acid and succinic acid); tricarboxylic acids (e.g. citric acid); phenolic compounds (e.g. resorcinol, catechol, phloroglucinol, salicylanilide, gallic acid and 2,4-dihydrobenzoic acid); polyacroleine; mono- and di-isocyanates (e.g. forming substituted carbamates); divinyl sulphate and other divinyl esters; and glycidyl and other difunctional methacrylates.

When the hair-removing composition comprises a mixture of a gel-promoting agent and polyvinyl alcohol, suitably the gel-promoting agent is present in an amount of between 0.01 wt % and 1 wt % of the total weight of the composition, preferably between 0.02 wt % and 0.5 wt %, more preferably between 0.05 wt % and 0.25 wt %.

Preferably when the hair-removing composition comprises polyvinyl alcohol the composition is an aqueous composition comprising water in an amount between 40 wt % and 90 wt %, preferably between 50 wt % and 70 wt % and more preferably between 50 wt % and 60 wt % of the total weight of the composition.

The amount of polyvinyl alcohol present in a preferred hair-removing composition of the invention, and the amount of a gel-promoting agent when present, will be in part determined by the desired viscoelastic properties of the composition. In order to further modify the viscosity of the depilatory composition, the composition may further comprise a polyhydric alcohol. Suitable polyhydric alcohols include glycerol, propylene glycol, mannitol, sorbitol, glucose, fructose, sucrose, propane diol and the like, but is preferably propylene glycol. Suitably the polyhydric alcohol is present in an amount of between 0.01 wt % and 50 wt %, preferably between 1 wt % and 40 wt % and preferably between 5 wt % and 30 wt % of the total weight of the composition.

When, as is preferred, boric acid is present as a gel-promoting agent, without being bound by any theory it is thought that polyvinyl alcohol reacts with the boric acid by a so-called “di-diol” complexation which is ion-assisted from the borate. Two diol units of PVA are thought to react with one borate ion to form a cross-link. It is thought that the PVA-borate crosslinking mechanism is divided into two sections; a mono-diol complexion and a cross-link formation.

Once a borate ion is attached to a polymer chain, it is believed to behave as a polyelectrolyte unless the borate ion is removed from the chain or is bound to another diol unit as a crosslinking point. In that case, a significant contribution of electrostatic repulsion between the monodiol units of the PVA chain is expected resulting in an extension of the individual polymer chains and the formation of a gel at a given concentration of PVA.

This phenomenon depends on the reactants, such as PVA concentration, borate ion concentration, and temperature.

A similar mechanism is thought to apply when other gel-promoting agents are employed.

“Boric acid” herein may be one or more of orthoboric (also known as boracic) acid, metaboric acid and tetraboric (also known as pyroboric) acid. Most preferred is orthoboric acid, H₃BO₃.

In a hair-removing composition having epilatory qualities, it is preferred that at low frequency of applied stress its viscous modulus exceeds its elastic modulus—so that it is liquid-like to put on—and at higher frequency of applied stress its elastic modulus exceeds its viscous modulus—so that it can be removed as a piece, e.g. by an abrupt peeling action. The point at which the viscous and elastic moduli are equal is called the crossover point (or gel point). Under reference conditions—temperature, 30° C.; applied stress 5 Pa; using an SR rheometer as described later—the crossover point is preferably at 1 rad/s or below, most preferably at 0.2 rad/s or below.

Preferably the value of the elastic and viscous moduli at the crossover point is less than 300 Pa, more preferably less than 100 Pa.

Preferably the elastic modulus above the crossover point is higher than the viscous modulus at all frequencies up to 10 rad/s, more preferably at all frequencies up to 100 rad/s; and may also be higher than the viscous modulus at higher frequencies.

Preferably the value of the elastic modulus is less than 300 Pa, more preferably less than 100 Pa, at all frequencies up to 10 rad/s, more preferably at all frequencies up to 100 rad/s.

Preferably the elastic modulus reaches a level or gently ascending plateau (the “rubbery plateau”) with rising frequency above the crossover point.

To summarise, below the crossover point the viscous property predominates and the composition is liquid-like and highly suitable for application to skin and hairs. Above the crossover point the elastic property predominates and the composition is rubber-like, and highly suitable for good removal, carrying with it removed hairs.

The elastic modulus G′ (sometimes known as the storage modulus) corresponds to the energy which can be stored and released by a bulk material. The viscous modulus G″ (sometimes known as the loss modulus) corresponds to the energy dissipated by a bulk material due to friction between its macromolecules when it is deformed.

$$\begin{matrix}{G}^{′} = \frac{{σ}_{∘}}{{γ}_{∘}} cos δ \\ {G}^{″} = \frac{{σ}_{∘}}{{γ}_{∘}} sin δ\end{matrix}$$

wherein σ. is the stress amplitude, γ. is the strain amplitude and δ is the out-of-phase coefficient.

The measurements quoted later are based on studies carried out into the rheology of the viscoelastic compositions in order to obtain a better understanding of their adhesive behaviour and their suitability as epilatory materials. These studies involved subjecting the materials to dynamic investigations in which a sinusoidal strain at defined frequencies was applied to the materials and the resulting output force was measured. In these studies a stress control rheometer was used, the SR rheometer commercially available from the company Rheometrics, using parallel plate geometry of 40 mm in diameter. The sample to be tested is squeezed between the plates, until there is a gap between the plates of 1 mm, and so a sample thickness of 1 mm. Material squeezed out from between the plates is removed.

The output force from the rheometer includes an in-phase elastic component G′ and an out-of-phase viscous component G″. The output force can be expressed as follows.

$$\begin{matrix}σ = {σ}_{∘} sin ( t ω + δ ) \\ = {σ}_{∘} cos δ sin t ω + {σ}_{∘} cos δ cos t ω\end{matrix}$$

where ω is the test frequency and t is the time.

The hair-removing composition, whether having an epilatory and/or depilatory property, may contain further ingredients such as a surfactant; a colorant; a fragrance or perfume; a filler such as talc, calcium carbonate or a fibrous material; a preservative; or a dye, as are conventionally included in cosmetic compositions.

Preferably the hair-removing composition, whether having an epilatory and/or depilatory property, is alkaline, more preferably of pH in the range 10 to 14, most preferably of pH in the range 11 to 13. An especially preferred pH range of the composition is between pH 12 and 12.5.

Suitably the composition comprises a pH regulator, to regulate the pH of the composition within the preferred ranges. Suitable pH regulators are bases, and may include arginine, especially L-arginine; silicates, especially sodium or potassium silicates; lime; polyethylenimine; potassium hydroxide; calcium hydroxide; lithium hydroxide; and sodium hydroxide.

The pH regulator is thought to assist in promoting the action of a depilatory agent, when present, through maintaining optimum pH for action of the agent on keratin/hair.

A particularly preferred hair-removing composition for use in a patch of the invention comprises an alkali metal (preferably potassium) hydroxide, lime, and an alkali metal (preferably potassium) thioglycolate, polyvinyl alcohol, boric acid and propylene glycol.

According to a second aspect of the invention there is provided a method of depilation comprising: (a) removing or displacing the closure member of the package as defined above; (b) removing the hair-removing layer from the product of the first aspect and applying it to the skin; and (c) removing the hair-removing layer from the skin.

Preferably the layer is depilatory and there is an intermediate step (b1) of leaving the layer on the skin for a prescribed period of time.

The prescribed period of time in step (b) will depend on the depilatory composition, but may typically be between 60 seconds and 1200 seconds, preferably 180 seconds and 900 seconds and more preferably between 300 seconds and 600 seconds.

According to a third aspect of the invention there is provided a method of manufacturing the product of the first aspect comprising the step of introducing hair-removing composition into the well as a warm, flowable composition and applying the closure member.

Preferably a product of the first aspect of the invention is protected from air until it is used. Preferably the container is airtight. The closure member is preferably in the form of a polymeric or metallic foil, or a metallised polymeric foil, preferably which is removable by a peeling action, in order to access the layer. The sheet is preferably of a gas-impermeable thermoplastics material.

The manufacturing method may thus comprise a step (c) of applying a closure member to enclose the layer within an airtight package, preferably enclosing a vacuum or inert atmosphere.

The invention will now be described further with reference to the following non-limiting examples.

The following materials were used in the examples:
Deionised water
Polyvinylalcohol (PVA)—PVA Mowiol 4-98 available, from Clariant
Boric acid—4% solution w/w in water
Propylene glycol
Potassium thioglycolate—30% w/w solution in water
Potassium hydroxide—a 50% w/w solution in water
Calcium hydroxide
Alcohol ethoxylate 11EO
Fragrance—floral tones

### EXAMPLE 1

The following ingredients/amounts were used:

| Ingredient | w/w total composition (%) |
|---|---|
| Deionised water | 56.18 |
| PVA | 6 |
| Boric acid (4%) | 0.12 |
| Propylene glycol | 22.1 |
| Potassium thioglycolate (30%) | 10 |
| Potassium hydroxide (50%) | 4.90 |
| Fragrance | 0.7 |

The required proportion of deionised water was heated to 80° C. in a reactor. The PVA was added and the mixture was stirred in the reactor vessel for the necessary time for all of the PVA to be dissolved. The mixture was then cooled down to 40° C. and a solution of boric acid was added to it. After 30 minutes of stirring, the propylene glycol was introduced with stirring, then the potassium thioglycolate, followed by the fragrance. Finally, the potassium hydroxide was introduced dropwise with stirring. The resulting solution was then transferred to a suitable storage container.

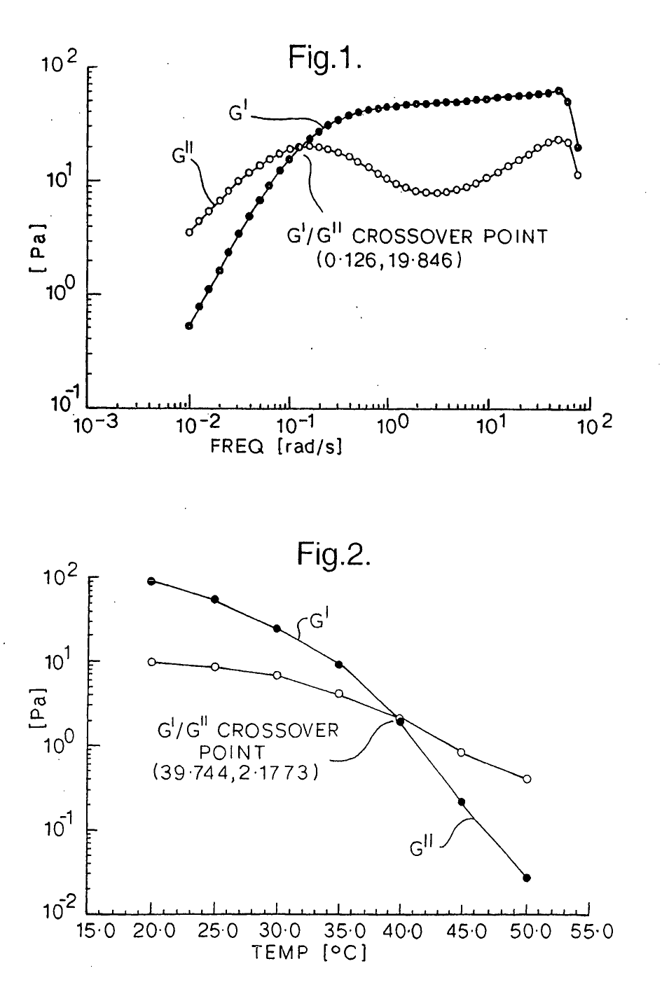

The resultant formulation had a pH of 12.5 and was in a form of a viscoelastic tacky hydrogel. Its mechanical properties, determined as described above, are shown in FIGS. 1 and 2.

Thus, measurements were made by an SR rheometer from rheometric, in a dynamic mode. G′ and G″ are respectively the elastic and viscous modulus characterising the tackiness of the gel.

FIG. 1 is a graph of G′ and G″ against frequency, as described above.

FIG. 2 is a graph of G′ and G″ against test temperature, under conditions of 5 Pa stress applied at 5 rad/s, with 5 minute test periods.

It will be seen from FIG. 1 that at low test frequencies the viscous modulus exceeds the elastic modulus. At higher test temperatures the elastic modulus exceeds the viscous modulus. The crossover point is at a modulus of 19.8 Pa and a frequency of 0.126 rad/s. Beyond the crossover point the elastic modulus follows a gently ascending plateau, as the test frequency increases. FIG. 2 shows that under the test conditions the elastic modulus exceeds the viscous modulus at all temperatures up to 39.7° C., the moduli then both being 2.18 Pa. This is a good indication that the patch will be suitable for use under all normal ambient conditions.

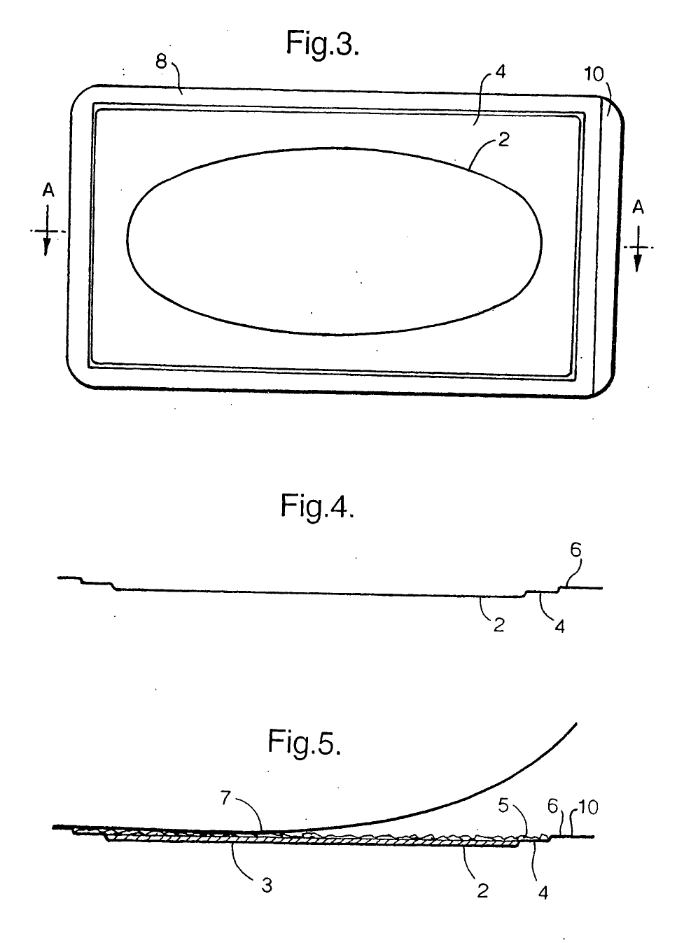

FIGS. 3, 4 and 5 show a patch intended for removal of axillary hair, and a package therefor. FIG. 3 is a plan view of a tray part of a package, not containing a patch. FIG. 4 is a side sectional view of the tray part of the package, not containing a patch, along view A-A′ shown in FIG. 3. FIG. 5 is a corresponding view, with the tray containing a patch.

As may be seen most clearly in FIG. 4 the package includes a moulded tray part having a central, wide, shallow, well 2, oval in plan view. The tray part is of a gas impermeable plastics material, manufactured as a sheet but shaped to form the tray part by thermoforming.

The warm composition of Example 1 is injected into the well 2, in a controlled amount such that it just fills the well and no more (see FIG. 5). Beyond the well 2, and extending all around it, is a ledge 4, of rectangular exterior shape. The non-woven textile substrate 5 of matching rectangular shape is laid onto the ledge 4 and over the depilatory composition 2, thereby forming the patch (see FIG. 5). Beyond this ledge 4 and extending all around it there is an uppermost ledge 6. The uppermost ledge is the periphery of the tray-like part and is rectangular, but with rounded corners. A release layer in the form of a closure foil 7, of metal, polymeric or metallised polymeric material is laid onto this uppermost ledge 6 so as to form a seal against it. As this happens the air in the package is flushed out with nitrogen. The seal is achieved in this embodiment by means of a heat-activated adhesive. The rectangular shaded zone 8 in FIG. 3 denotes the seal. The seal is of a high quality; air cannot pass it to enter the package.

The combination of the gas-impermeable tray and foil, the impermeable seal between them and the exclusion of oxygen within the package all serve to shield the depilatory composition—in particular the potassium thioglycolate component—from the degradative effects of the oxygen in the external atmosphere.

It will be seen that at one end of the package there is a region 10 beyond the seal. In this region 10 the uppermost ledge 6 is wider than elsewhere and the foil is adhered only to an inner band of this widened ledge. Thus, in this region the foil may be gripped by the user in order to tear it away, allowing the user to remove the patch. In FIG. 5 the foil 7 is shown partly removed. When it is removed the non-woven substrate 5 may be grasped at its periphery in order to lift the patch from the package. The patch may then be pressed onto an axilla.

In this embodiment the smallest diameter of the well, that is, of depilatory layer, is 65 mm. The largest diameter is 145 mm. The size of the substrate is 170 mm×90 mm. The size of the pack is 192 mm by 105 mm. The outer ledge 6 is 7.5 mm wide around three sides but the other side, having the foil edge which can be grasped for peeling away, is 14.5 mm wide.

The liquid-like properties at low frequency assist the wetting process. The patch is comfortable to wear. The substrate prevents undesirable migration of the depilatory composition.

After 5 minutes the patch is lifted off, wherein the hair previously present on the skin is found to be substantially completely removed, and hair debris can be seen on the depilatory patch. The act of peeling off the patch gives a mild epilatory effect, carrying off hairs which have been thinned by the depilatory agent. The act of peeling off the patch corresponds to a higher frequency condition in which the elastic modulus predominates, aiding the clean removal of the patch as a rubber-like body, with the mild epilatory effect mentioned above.

### EXAMPLE 2

The following ingredients/amounts were used:

| Ingredient | w/w total composition (%) |
|---|---|
| PVA | 4 |
| Deionised water | 59.88 |
| Boric acid (4%) | 0.12 |
| Thioglycolic acid (30%) | 10 |
| Propylene glycol | 22.1 |
| Fragrance | 0.7 |
| Alcohol ethoxylate 11EO | 1.5 |
| Calcium hydroxide | 1.7 |
| Total | 100.00% |

They were used to make a depilatory composition also having some epilatory properties, in the manner described in Example 1, for conjoining to a substrate to form a depilatory patch.

### EXAMPLE 3

The following ingredients/amounts were used:

| Ingredient | w/w total composition (%) |
|---|---|
| PVA | 8 |
| Deionised water | 55.88 |
| Boric acid (4%) | 0.12 |
| Thioglycolic acid (30%) | 10 |
| Propylene glycol | 22.1 |
| Fragrance | 0.7 |
| Alcohol ethoxylate 11EO | 1.5 |
| Calcium hydroxide | 1.7 |
| Total | 100.00% |

They were used to make a depilatory composition also with some epilatory properties in the manner described in Example 1, for conjoining to a substrate to form a depilatory patch.

1. A packaged hair removing product comprising a container and a hair-removing layer therein, wherein the container comprises a sheet formed with a shallow well, the hair-removing layer being retained in the shallow well, the container further comprising a closure member which retains the hair-removing layer in the container, the closure member being removable or displaceable from the container to permit removal of the hair-removing layer, wherein the hair-removing layer has a depilatory action and said layer is in inseparable contact with a substrate, to form a patch.

2. A product according to claim 1, wherein the substrate projects laterally from the layer and the sheet has a stepped recess by accommodate snugly the layer and the projection of the substrate beyond the layer.

3. A product according to claim 1, wherein the sheet has a flange or rim around the whole of its periphery against which the closure member is sealed.

4. A product according to claim 1, wherein the layer comprises a polymeric material.

5. A product according to claim 4, wherein the polymeric material comprises polyvinyl alcohol.

6. A product according to claim 5, wherein a cosmetically acceptable gel-promoting agent is employed with the polyvinyl alcohol.

7. A product according to claim 6, wherein the cosmetically acceptable gel-promoting agent is a borate or boric acid.

8. A product according to claim 5, wherein the depilatory composition is an aqueous composition comprising water in an amount between 40 wt % and 90 wt %, of the total weight of the composition.

9. A product according to claim 5, wherein the composition further comprises a polyhydric alcohol.

10. A product according to claim 1, wherein the depilatory composition is alkaline.

11. A method of depilation comprising (a) removing or displacing a closure member of a product according to claim 1, to access the layer; (b) applying the layer to the skin; and (c) removing the layer from the skin.

12. A method of manufacturing a product according to claim 1, comprising the steps of: (a) introducing the hair-removing composition as a warm, flowable composition into the shallow well; and (b) closing the container by means of the closure member.

13. (canceled)

14. (canceled)

15. (canceled)

16. A product according to claim 6 wherein the depilatory composition is an aqueous composition comprising water in an amount between 40 wt % and 90 wt %, of the total weight of the composition.

17. A product according to claim 7 wherein the depilatory composition is an aqueous composition comprising water in an amount between 40 wt % and 90 wt %, of the total weight of the composition.

18. A product according to claim 6 wherein the composition further comprises a polyhydric alcohol.

19. A product according to claim 7 wherein the composition further comprises a polyhydric alcohol.

20. A product according to claim 8 wherein the composition further comprises a polyhydric alcohol.
